(19) United States
(12) Patent Application Publication
(10) Pub. No.: US 2020/0337542 A1
(43) Pub. Date: Oct. 29, 2020

(54) HYBRID IMAGING PRODUCT AND HYBRID ENDOSCOPIC SYSTEM

(71) Applicant: CHEMIMAGE CORPORATION, Pittsburgh, PA (US)

(72) Inventors: Shona STEWART, Pittsburgh, PA (US); Patrick J. TREADO, Pittsburgh, PA (US); Alyssa ZRIMSEK, Pittsburgh, PA (US); Jihang WANG, Sewickley, PA (US)

(21) Appl. No.: 16/859,589

(22) Filed: Apr. 27, 2020

Related U.S. Application Data
(60) Provisional application No. 62/839,220, filed on Apr. 26, 2019.

(51) Int. Cl. A61B 1/07; A61B 1/00; A61B 1/06; A61B 1/05

(52) U.S. Cl. CPC A61B 1/07; A61B 1/05; A61B 1/0684; A61B 1/00096

(57) ABSTRACT

An endoscope has an improved chip-on-tip configuration that includes both a first configuration of source illumination fibers and a second plurality of source illumination fibers, along with a camera chip. The combination of the camera chip and the source illumination fibers on the tip of the endoscope results in endoscopes with reduced size and weight.

### CROSS-REFERENCE TO RELATED APPLICATIONS

This application claims priority to U.S. Provisional Application Ser. No. 62/839,220, the entire contents of which are incorporated by reference herein.

### FIELD

The present disclosure relates generally to various sensor and illumination configurations for endoscopy. More specifically, the present disclosure relates to various sensor and illumination configurations for chip-on-tip endoscopy.

### BACKGROUND

Endoscopes have attained great acceptance within the medical community because they allow procedures to be performed with minimal patient trauma while enabling the physician to view the internal anatomy of the patient. Depending upon the procedure, an endoscope may be inserted into a body's natural orifices or through an incision in the skin.

Conventional endoscope designs typically include an elongated tubular shaft having a rigid lens assembly or fiber optic lens assembly at one end connected to a camera or other similar light sensor via the rigid lens assembly or one or more fiber optic strands. The shaft is connected to a handle for manipulation during a procedure. Viewing is usually possible via an ocular lens in the handle and/or via an external screen. Various surgical tools may be inserted through a working channel in the endoscope for performing different surgical procedures.

Applicant previously disclosed chip-on-tip endoscope configurations in U.S. Application Ser. No. 61/615,777 filed 10 Jan. 2018 and entitled “TIME CORRELATED SOURCE MODULATION FOR ENDOSCOPY”, U.S. application Ser. No. 16/244,845 filed 10 Jan. 2019 and entitled “TIME CORRELATED SOURCE MODULATION FOR ENDOSCOPY”, and World Intellectual Property Organization Application Serial No. PCT/US2019/013067 filed 10 Jan. 2019 and entitled “TIME CORRELATED SOURCE MODULATION FOR ENDOSCOPY”, each of the foregoing applications is incorporated by reference herein in its entirety.

Chip-on-tip endoscope configurations differ from conventional endoscope configurations because the camera is at the tip of the endoscope rather than at the base. There are several benefits to chip-on-tip configurations. For example, by placing the camera as close to the sample as possible, throughput losses for the signal are reduced and image distortion through lens assemblies and fiber optics is minimized. In addition, chip-on-tip endoscopes can result in systems that weigh less and are smaller than typical endoscopes. Because the signal is read onto the camera chip at the tip of the endoscope, specialized optics or fiber optics are not required to relay the image to a camera at the back of the endoscope. As such, the overall number of components in the endoscopic system is reduced.

Despite this, conventional endoscopes retain certain advantages. For example, complex imaging hardware generally cannot fit on an endoscope tip and optical fibers are required to transmit images from the distal end of the endoscope at the patient or tissue sample to the proximal end of the endoscope where the imaging hardware is located.

As such, there is a need for a hybrid endoscope that combines the advantages of chip-on-tip endoscope configurations with the advantages of conventional endoscope designs by combining the different elements of each design in novel, synergistic ways.

### SUMMARY

The disclosure is directed to various embodiments of chip-on-tip products that are used with or within an endoscope.

In some embodiments, a hybrid imaging product for use in an endoscope is disclosed, the hybrid imaging product including a first plurality of source illumination fibers configured to transmit a first plurality of modulated photons; a second plurality of source illumination fibers configured to transmit a second plurality of modulated photons; a plurality of fiber array spectral translator (FAST) fibers; and a first image collector that includes one or more of imaging fibers or a camera sensor. In some embodiments, the hybrid imaging product further includes a third plurality of source illumination fibers configured to transmit a third plurality of unmodulated photons. In some embodiments, the hybrid imaging product further includes a second image collector, and the second image collector includes one or more of imaging fibers or a camera sensor.

In some embodiments, an endoscopic system includes a hybrid imaging product that includes a first plurality of source illumination fibers configured to transmit a first plurality of modulated photons, a second plurality of source illumination fibers configured to transmit a second plurality of modulated photons, a plurality of fiber array spectral translator (FAST) fibers, and a first image collector that includes one or more of imaging fibers or a camera sensor; an illumination source; a first modulator that modulates at least the first plurality of modulated photons for the first plurality of source illumination fibers; and a second modulator that modulates at least the second plurality of modulated photons for the second plurality of source illumination fibers. In some embodiments, the hybrid imaging product further comprises a third plurality of source illumination fibers configured to transmit a third plurality of unmodulated photons. In some embodiments, the hybrid imaging product further includes a second image collector, and the second image collector includes one or more of imaging fibers or a camera sensor. In some embodiments, the illumination source includes an incandescent lamp, halogen lamp, light emitting diode (LED), quantum cascade laser, quantum dot laser, external cavity laser, chemical laser, solid state laser, organic light emitting diode (OLED), electroluminescent device, fluorescent light, gas discharge lamp, metal halide lamp, xenon arc lamp, induction lamp, or combinations thereof. In some embodiments, the first modulator or the second modulator is each independently one or more of an acousto-optic tunable filter (AOTF), a liquid crystal tunable filter (LCTF), a multivariate optical element (MOE), a filter wheel, a patterned etalon filter, a multi-conjugate filter (MCF), or a conformal filter (CF).

In some embodiments, a method of generating a fused image using an endoscopic system that includes a hybrid imaging product that includes a first plurality of source illumination fibers configured to transmit a first plurality of modulated photons, a second plurality of source illumination fibers configured to transmit a second plurality of modulated photons, a plurality of fiber array spectral translator (FAST) fibers, and a first image collector that includes one or more of imaging fibers or a camera sensor; an illumination source; a first modulator that modulates at least the first plurality of modulated photons for the first plurality of source illumination fibers; and a second modulator that modulates at least the second plurality of modulated photons for the second plurality of source illumination fibers, the method comprising generating a first image from a first plurality of modulated photons; generating a second image from a second plurality of modulated photons; and overlaying the first image and the second image to thereby generate a fused image. In some embodiments, the method further comprises generating a third image from a third plurality of unmodulated photons. In some embodiments, the third plurality of unmodulated photons are NIR photons, SWIR photons, eSWIR photons, or combinations thereof. In some embodiments, each of the first plurality of modulated photons and the second plurality of modulated photons are independently VIS or VIS-NIR.

### DRAWINGS

The accompanying drawings, which are incorporated in and form a part of the specification, illustrate the embodiments of the invention and together with the written description serve to explain the principles, characteristics, and features of the invention. In the drawings:

FIG. 1 illustrates a first variation of an endoscope in accordance with the present disclosure.

FIG. 2 illustrates a second variation of an endoscope in accordance with the present disclosure.

FIG. 3 illustrates a third variation of an endoscope in accordance with the present disclosure.

FIG. 4 illustrates two optical configuration options for use with one of the endoscope variations as shown in FIGS. 1-3 in accordance with the present disclosure.

FIG. 5 illustrates a fourth variation of an endoscope in accordance with the present disclosure.

FIG. 6 illustrates a fifth variation of an endoscope in accordance with the present disclosure.

FIG. 7 illustrates a sixth variation of an endoscope in accordance with the present disclosure.

FIG. 8 illustrates an embodiment of a Fiber Array Spectral Translator device in accordance with the present disclosure.

FIG. 9 illustrates an embodiment of a hybrid endoscope in accordance with the present disclosure.

### DETAILED DESCRIPTION

This disclosure is not limited to the particular systems, devices and methods described, as these may vary. The terminology used in the description is for the purpose of describing the particular versions or embodiments only, and is not intended to limit the scope.

As used in this document, the singular forms “a,” “an,” and “the” include plural references unless the context clearly dictates otherwise. Unless defined otherwise, all technical and scientific terms used herein have the same meanings as commonly understood by one of ordinary skill in the art. Nothing in this disclosure is to be construed as an admission that the embodiments described in this disclosure are not entitled to antedate such disclosure by virtue of prior invention. As used in this document, the term “comprising” means “including, but not limited to.”

The embodiments described below are not intended to be exhaustive or to limit the teachings to the precise forms disclosed in the following detailed description. Rather, the embodiments are chosen and described so that others skilled in the art may appreciate and understand the principles and practices of the present teachings.

As noted above, conventional endoscopes include a fiber optic lens assembly disposed at one end connected to a camera or other similar light sensor via one or more fiber optic strands. In order to acquire multispectral data from a conventional endoscopic imaging system, the signal acquired from a sample is tuned selectively, often by one or more in-line filters, before reaching the camera. In endoscopic systems with the camera (or chip) on the tip of the endoscope, it is not readily possible without significant miniaturization of filtering technology to filter the sample image. Instead, the source illumination is filtered before reaching the sample. The sample signal is then read by the camera at the tip of the endoscope.

This disclosure describes several endoscopic imaging variations. These variations include filtering the source illumination prior to sample imaging. In each of the variants, source filtering and/or modulation can be performed by, for example, an optical imaging filter such as an acousto-optic tunable filter (AOTF), a liquid crystal tunable filter (LCTF), and/or a sequential scan tunable filter including, for example, a multi-conjugate filter (MCF) or conformal filter (CF). It should be noted, however, that MCFs and CFs are described herein by way of example only. Additional filters such as multivariate optical elements (MOEs), MOE filter and filter wheel arrangements, patterned etalon filters, and other similar filters can be used to filter the source illumination. Additional examples of source illumination processing and filtering can be found in U.S. patent application Ser. No. 15/374,769 which is published as U.S. Patent Application Publication No. 2018/0116494, the content of which is incorporated herein by reference in its entirety. The above source filtering and/or modulation is denoted in some embodiments as being performed by a “modulator,” which refers to any of the devices that modulate photons from the illumination source.

As described herein, the variations comprise the camera chips placed in the center of the endoscope tip and surrounded by the source illumination fibers. However, it should be noted that this central arrangement is provided by way of example only, and additional arrangements of the camera chips and the source illumination fibers can be included.

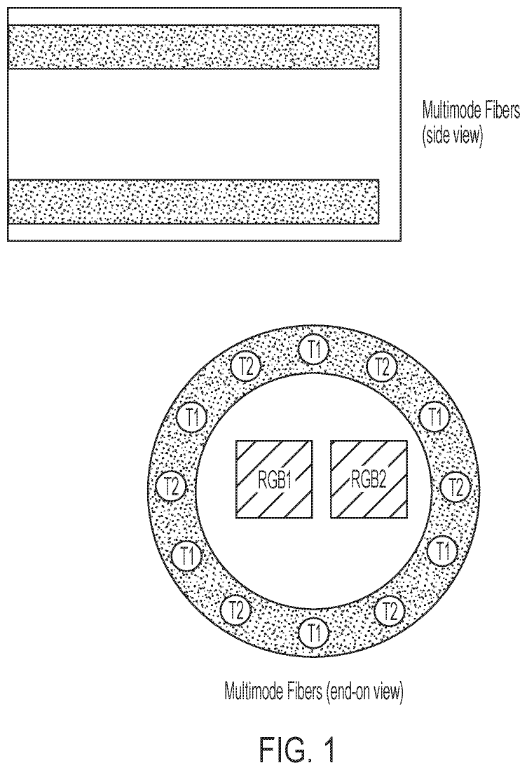

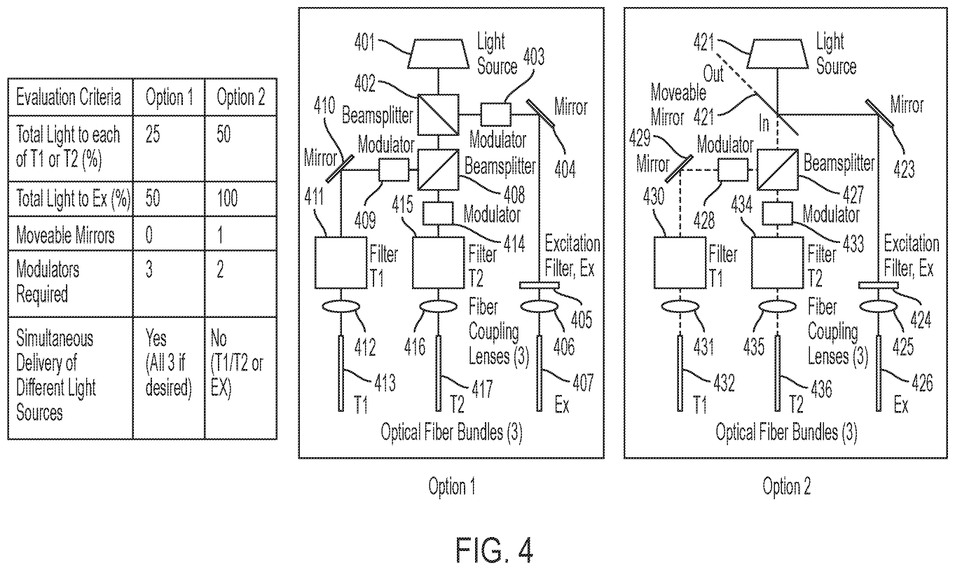

The endoscope variations as described herein can include various illumination sources. In certain implementations, a single illumination source can be used in combination with various configurations of beamsplitters and/or mirrors to provide multiple light beams. These multiple light beams can then be directed to the tip of the endoscope using, for example, different source illumination fibers or sets of source illumination fibers. For example, in the variation shown in FIG. 1, a single illumination source can be split into two separate beams and directed to two sets of source illumination fibers. In other implementations, such as the options illustrated in FIG. 4, a single illumination source can be split into separate beams, or redirected via one or more mirrors, to provide source illumination for three sets of source illumination fibers. It should be noted that although only a single illumination source is shown, for example, in FIGS. 1 and 4, the disclosure is not limited to a single illumination source. It is contemplated that additional illumination sources can be included in one or more of the endoscope variations as described herein. Such endoscope variations can include a plurality of separate and distinct illumination sources used alone or in combination.

The number of illumination fibers is not limited. In some embodiments, the number of illumination fibers is about 50, about 60, about 70, about 80, about 90, about 100, about 110, about 120, about 130, about 140, about 150, about 200, about 300, about 400, about 500, about 600, about 700, about 800, about 900, about 1000, about 1100, about 1200, about 1300, about 1400, about 1500, or a number of fibers in a range between any two of the above.

The illumination source is not limited and can be any source that is useful in providing the necessary illumination for the endoscope other ancillary requirements, such as power consumption, emitted spectra, packaging, thermal output, and so forth. In some embodiments, the illumination source is an incandescent lamp, halogen lamp, light emitting diode (LED), quantum cascade laser, quantum dot laser, external cavity laser, chemical laser, solid state laser, organic light emitting diode (OLED), electroluminescent device, fluorescent light, gas discharge lamp, metal halide lamp, xenon arc lamp, induction lamp, or any combination of these illumination sources. In some embodiments, the illumination source is a tunable illumination source, which means that the illumination source is monochromatic and can be selected to be within any desired wavelength range. The selected wavelength of the tunable illumination source is not limited and can be any passband within the ultraviolet (UV), visible (VIS), near infrared (NIR), visible-near infrared (VIS-NIR), shortwave infrared (SWIR), extended shortwave infrared (eSWIR), and near infrared-extended shortwave infrared (NIR-eSWIR) ranges. The wavelength ranges are described below.

The disclosed variations of the endoscopes include at least one camera chip that is used as an image sensor to detect incoming photons and output that information to form an image. The functionality and construction of the camera chip is not limited. In some embodiments, the camera chip is characterized by the wavelengths of light that it is capable of imaging. The wavelengths of light that can be imaged by the camera chip are not limited, and include ultraviolet (UV), visible (VIS), near infrared (NIR), visible-near infrared (VIS-NIR), shortwave infrared (SWIR), extended shortwave infrared (eSWIR), near infrared-extended shortwave infrared (NIR-eSWIR). These classifications correspond to wavelengths of about 180 nm to about 380 nm (UV), about 380 nm to about 720 nm (VIS), about 400 nm to about 1100 nm (VIS-NIR), about 850 nm to about 1800 nm (SWIR), about 1200 nm to about 2450 nm (eSWIR), and about 720 nm to about 2500 nm (NIR-eSWIR). The above ranges may be used alone or in combination of any of the listed ranges. Such combinations include adjacent (contiguous) ranges, overlapping ranges, and ranges that do not overlap. The combination of ranges may be achieved by the inclusion of multiple camera chips, each sensitive to a particular range, or a single camera chip that by the inclusion of a color filter array can sense multiple different ranges.

In some embodiments, the camera chip is characterized by the materials from which it is made. The materials of the camera chip are not limited and can be selected based on the wavelength ranges that the camera chip is expected to detect. In such embodiments, the camera chip comprises silicon (Si), germanium (Ge), indium gallium arsenide (InGaAs), platinum silicide (PtSi), mercury cadmium telluride (HgCdTe), indium antimonide (InSb), colloidal quantum dots (CQD), or combinations of any of these.

In some embodiments, the camera chip is characterized by its electrical structure. The electrical structure is not limited. In some embodiments, the camera chip includes a charge coupled device (CCD) or a complementary metal oxide semiconductor (CMOS) image sensor. It should be noted that the materials listed above can each be used with either electrical structure to form the final camera chip. Examples include Si CCD, Si CMOS, Ge CCD, Ge CMOS, InGaAs CCD, InGaAs CMOS, PtSi CCD, PtSi CMOS, HgCdTe CCD, HgCdTe CMOS, InSb CCD, InSb CMOS, CQD CCD, and CQD CMOS. These sensor structures may be used alone or in combination, either in the same physical camera chip or in multiple separate camera chips.

In some embodiments, the camera chip is provided with a color filter array to produce images. The design of the filter array is not limited. It is to be understood that the term “filter” when used in the context of a camera chip means that the referenced light is allowed to pass through the filter. For example, a “green filter” is a filter that appears green to the human eye by only allowing light having a wavelength of about 520 nm to about 560 nm to pass through the filter, corresponding to the visible color green. A similar “NIR filter” only permits near infrared light (NIR) to pass through. In some embodiments, the filter is a color filter array that is positioned over a camera chip. Such color filter arrays are varied in design but are all related to the original “Bayer” filter color mosaic filters. The color filter array includes BGGR, RGBG, GRGB, RGGB, RGBE, CYYM, CYGM, RGBW (2×2), RGBW (2×2 with diagonal colors), RGBW (2×2 with paired colors), RGBW (2×2 with vertical W), and X-TRANS (sold by Fujifilm Corporation of Tokyo, Japan). The X-TRANS sensor has a large 6×6 pixel pattern that reduces Moiré effect artifacts by including RGB tiles in all horizontal and vertical lines. In the listings, B corresponds to blue, G to green, R to red, E to emerald, C to cyan, Y to yellow, and M to magenta. W corresponds to a “white” or a monochrome tile, which will be further described below.

The W or “white” tile itself includes several configurations. In some embodiments, the W tile does not filter any light, and so all light reaches the camera chip. In those embodiments, the camera chip will detect all of the light within a given range of wavelengths. Depending on the camera chip, this can be UV, VIS, NIR, VIS-NIR, VIS-NIR, VIS-SWIR, or VIS-eSWIR. In some embodiments, the W tile is a filter for VIS, VIS-NIR, NIR, or eSWIR, allowing only VIS, VIS-NIR, NIR, or eSWIR respectively to reach the camera chip. This may be advantageously combined with any of the camera chip materials or electrical structures listed above. Such a filter array can be useful because it enables a single camera chip to detect both visible light and near infrared light and is sometimes referred to as a four-band filter array.

In still further embodiments, the color filter array is omitted and is not provided with the camera chip, which produces a monochromatic image. In such embodiments, the generated image is based solely on the band gap of the materials that make up the camera chip. In other embodiments, a filter is still applied to the camera chip, but only as a monolithic, single filter. For example, the application of a red filter means that the camera chip generates monochromatic images representative of red spectrum. In some embodiments, multiple camera chips, each with a different monolithic, single filter camera chip are employed. As an example, a VIS image can be produced by combining three camera chips having R, G, and B filters, respectively. In another example, a VIS-NIR image can be produced by combining four camera chips having R, G, B, and NIR filters, respectively. In another example, a VIS-eSWIR image can be produced by combining four camera chips having R, G, B, and eSWIR filters, respectively.

In some embodiments, the color array is omitted, and the camera chip utilizes vertically stacked photodiodes organized into a pixel grid. Each of the stacked photodiodes responds to the desired wavelengths of light. For example, a stacked photodiode camera chip includes R, G, and B layers to form a VIS image. In another embodiment, the stacked photodiode camera chip includes R, G, B, and NIR layers to form a VIS-NIR image. In another embodiment, the stacked photodiode camera chip includes R, G, B, and eSWIR layers to form a VIS-eSWIR image.

In those embodiments where two or more camera chips are included, a stereoscopic image may be generated based on the images from each of the two or more camera chips. Stereoscopic images are useful because they permit a viewer to perceive depth in the image, which results in improved accuracy and realism over monoscopic images. During surgery or other similar endoscopic activities, stereoscopic images are useful for manipulating instruments and performing tasks, with greater safety and accuracy than with monoscopic endoscopes. This is because monoscopic endoscopes, having only one camera chip position, cannot provide depth perception. In some embodiments, the stereoscopic image is formed by using two camera chips and two color filter arrays that are the same. In some embodiments, the stereoscopic image is formed by two camera chips that are the same, but each provided with a different color filter array. In some embodiments, the stereoscopic image is formed by two camera chips that are different, provided with two color filter arrays that are different. In some embodiments, the stereoscopic image is formed by two camera chips that are different, with one camera chip being provided with a color filter array and the other camera chip being provided either a monochromatic filter or no filter array. If more than one camera chip is provided, a stereoscopic image can be generated by using the output of each camera chip and combining or fusing the output of each camera chip.

In still further embodiments, methods of obtaining stereoscopic images are provided. For example, a first camera chip generates a first image, a second camera chip at a different position generates a second image, and the first image and the second image are combined (“fused”) to form a stereoscopic image. Although two camera chips are described in these embodiments, it is understood that the total number of camera chips is not limited and can be increased to a total number greater than two. In some embodiments, there are third, fourth, fifth, or sixth camera chips.

FIG. 1 illustrates a first endoscope variation in accordance with the present disclosure. As shown in FIG. 1, an endoscope can be equipped with two red-green-blue (RGB) cameras RGB1 and RGB2 in the tip, surrounded by two sets of source illumination fibers T1 and T2. As shown in the end-on view in FIG. 1, the source illumination fibers T1, T2 can be arranged in an alternating manner around the circumference of the endoscope. Such an arrangement can provide for more uniform and consistent lighting around the circumference of the endoscope. However, it should be noted that the alternating arrangement is provided by way of example only, and additional arrangements of the source illumination fibers can be included in the design.

In some implementations, the source illumination fibers T1 and T2 can represent two discrete wavelengths filtered through sequential scan MCFs or a plurality of wavelengths filtered through CFs. For example, T1 and T2 can be modulated selectively or delivered to the sample simultaneously. As shown in the circuit diagram included in FIG. 1, a single illumination source can be directed at a beamsplitter. The beamsplitter can be configured to split the light received from the illumination source into two beams. A first beam can be directed down a T1 source illumination path to a modulator and filter (e.g., an MCF and/or a CF). Similarly, a second beam can be directed down a T2 source illumination path to a second modulator and filter (e.g., an MCF and/or a CF). By actively controlling the operation of the modulators and configuration of the filters, either or both of the first and second plurality of source illumination fibers T1 or T2 can illuminate a sample tissue at their respective discrete wavelengths.

In some implementations, the two cameras RGB1 and RGB2 can be configured to perform separate imaging functions. For example, RGB1 can be tuned and configured to provide sample images when the sample tissue is illuminated using source illumination fibers T1. Conversely, RGB2 can be tuned and configured to provide sample images when the sample tissue is illuminated using source illumination fibers T2. In other implementations, RGB1 may be implemented as a low-resolution camera, and RGB2 may be implemented as a high-resolution camera. In such an example, both cameras may be configured to capture images using either of source illumination fibers T1 and T2.

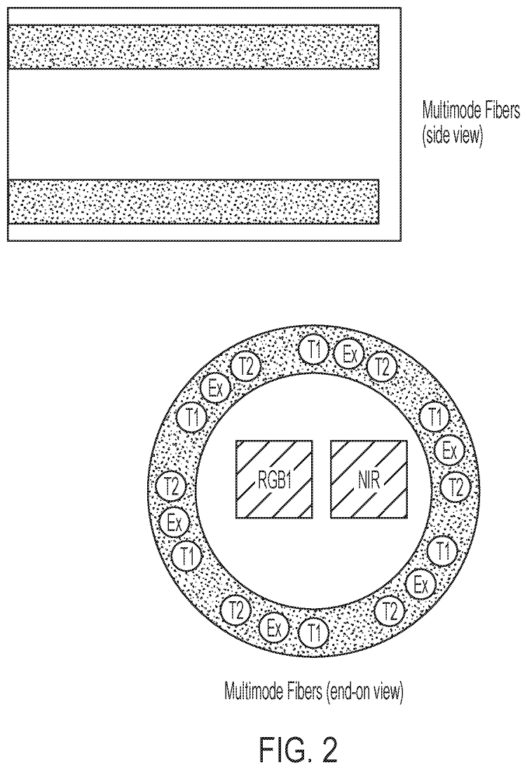

FIG. 2 illustrates a second endoscope variation in accordance with the present disclosure. As shown in FIG. 2, an endoscope can be equipped with one RGB camera and one near infrared (NIR) camera. Several fibers including source illumination fibers T1 and T2 (similar to T1 and T2 as described above), and a third source illumination fiber, Ex, can be arranged such that they surround the two camera chips. As described herein, source illumination fiber Ex can be configured to direct light which has an excitation wavelength for NIR fluorescence imaging. In still other embodiments, the third source illumination fibers can transmit one or more of ultraviolet (UV), visible (VIS), near infrared (NIR), or visible-near infrared (VIS-NIR) light. In still other embodiments, the light that is transmitted is monochromatic. In some implementations, the illumination fibers can be arranged in multiple groups of three. For example, as shown in the end-on view in FIG. 2, the groups of three can alternate between T1, Ex, and T2. However, it should be noted that the alternating arrangement shown in FIG. 2 is provided by way of example only. Alternate and/or additional arrangements of the source illumination fibers can be used in the design.

Similar to the above discussion of FIGS. 1, T1 and T2 can represent two discrete wavelengths filtered through sequential scan MCFs or a plurality of wavelengths filtered through CFs. In some implementations, all three illumination sources T1, T2, and Ex can be presented simultaneously to the sample. In some examples, illumination sources T1 and T2 can be delivered independently from illumination source Ex.

In some implementations, an image of a tissue sample illuminated using the T1 and T2 illumination source fibers can be recorded using the RGB camera. In such an arrangement, a fluorescence image of the tissue sample illuminated using the Ex source illumination fibers can be recorded using the NIR camera. However, it should be noted that such an arrangement is provided by way of example only, and the functionality of the cameras can be altered based upon the modulation, filtering, and other similar factors related to the source illumination fibers.

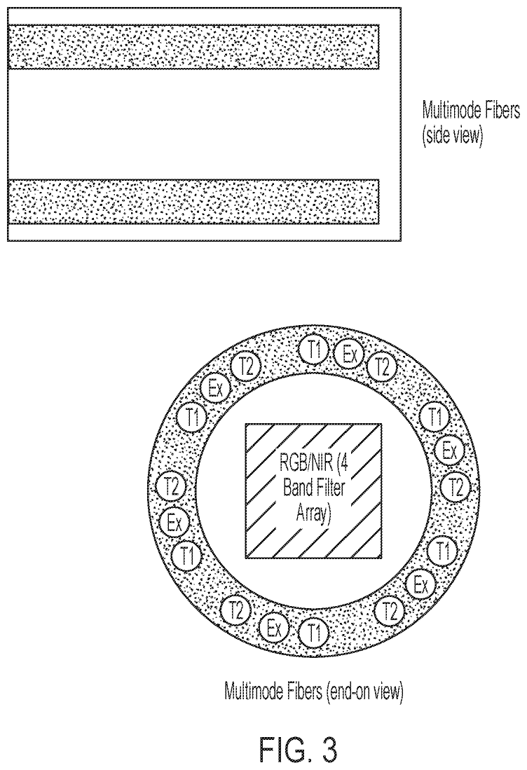

FIG. 3 illustrates a third endoscope variation in accordance with the present disclosure. As shown in FIG. 3, an endoscope can be equipped with a four-band filter array comprising red, green, blue, and NIR filters over the camera chip. Similar to the arrangement as described above in regard to FIG. 2, several fibers including source illumination fibers T1 and T2, and a third source illumination fiber, Ex, may surround the camera chip(s). In some implementations, the illumination fibers can be arranged in multiple groups of three. For example, as shown in the end-on view in FIG. 3, the groups of three can alternate between T1, Ex, and T2. However, it should be noted that the alternating arrangement shown in FIG. 3 is provided by way of example only. Alternate and/or additional arrangements of the source illumination fibers can be used in the design.

Similar to the above discussion of FIGS. 1, T1 and T2 can represent two discrete wavelengths filtered through sequential scan MCFs or a plurality of wavelengths filtered through CFs. In some implementations, all three illumination sources T1, T2, and Ex can be presented simultaneously to the sample. In some examples, illumination sources T1 and T2 can be delivered independently from illumination source Ex.

In some implementations, tissue samples imaged using illumination source fibers T1 and T2 can be recorded using the red, green, and/or blue filtered pixels of the filter array. In such an arrangement, fluorescence images generated using illumination source fibers Ex can be recorded using the NIR filtered pixels.

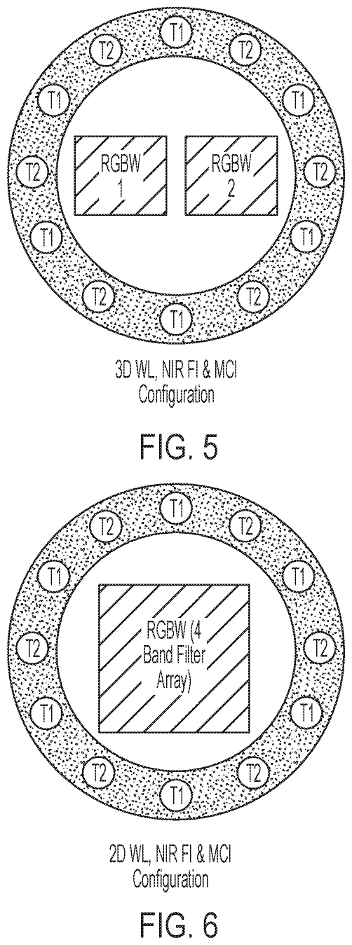

FIG. 5 illustrates yet another endoscope variation in accordance with the present disclosure. As shown in FIG. 5, an endoscope is equipped with two four-band filter arrays corresponding to two camera chips. Each four-band filter array comprises red, green, blue, and white (monochrome) filters, and each four-band filter array is placed over a separate camera chip. Similar to the arrangement as described above in regard to FIGS. 1, T1 and T2 can represent two discrete wavelengths filtered through sequential scan MCFs or a plurality of wavelengths filtered through CFs. The use of two separate camera chips results in a stereoscopic image. In some embodiments (not shown), all three illumination sources T1, T2, and Ex can be presented simultaneously to the sample. In some examples, illumination sources T1 and T2 can be delivered independently from illumination source Ex.

FIG. 6 illustrates yet another endoscope variation in accordance with the present disclosure. As shown in FIG. 6, an endoscope is equipped with a single four-band filter array corresponding to a single camera chip. The four-band filter array comprises red, green blue, and white (monochrome) filters. Similar to the arrangement as described above in regard to FIGS. 1, T1 and T2 can represent two discrete wavelengths filtered through sequential scan MCFs or a plurality of wavelengths filtered through CFs. In some embodiments (not shown), all three illumination sources T1, T2, and Ex can be presented simultaneously to the sample. In some examples, illumination sources T1 and T2 can be delivered independently from illumination source Ex.

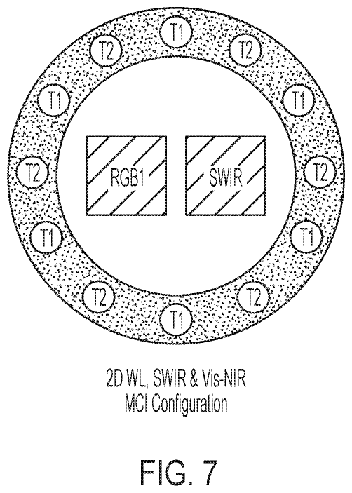

FIG. 7 illustrates yet another endoscope variation in accordance with the present disclosure. As shown in FIG. 7, an endoscope comprises a RGB filter that is depicted as RGB1 and that is positioned on a first camera chip. The endoscope of FIG. 7 also comprises a SWIR filter, and the SWIR filter is positioned on a second camera chip. Similar to the arrangement described above in regard to FIGS. 1, T1 and T2 can represent two discrete wavelengths filtered through sequential scan MCFs or a plurality of wavelengths filtered through CFs. In some embodiments (not shown), all three illumination sources T1, T2, and Ex can be presented simultaneously to the sample. In some examples, illumination sources T1 and T2 can be delivered independently from illumination source Ex. With respect to FIG. 7, it is appreciated that although two physically separate camera chips are used, only a two dimensional or non-stereoscopic image is generated in white or RGB light that is visible to the human eye, because the SWIR filter is positioned on the second camera chip and cannot contribute to forming a stereoscopic image.

In some embodiments, the endoscopic system further includes a plurality of fibers that are part of a fiber array spectral translator (FAST) device. Fibers that are part of a fiber array spectral translator (FAST) device are referred to herein as “FAST fibers.” In some embodiments, the FAST fibers are included within the body of the endoscope. When the FAST fibers are included within the body of the endoscope, the endoscope can simultaneously image with the camera chips and the FAST fibers.

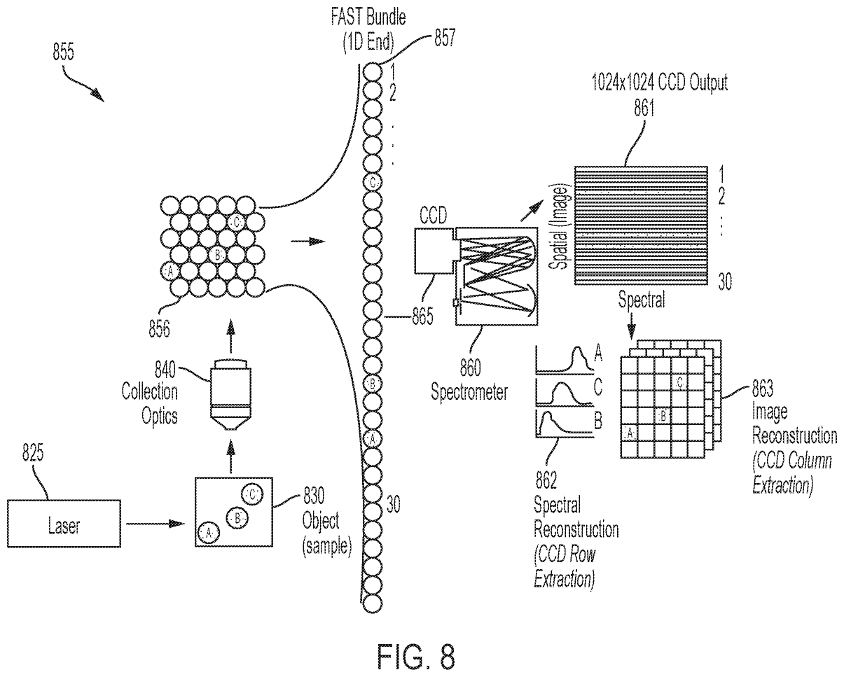

FIG. 8 illustrates an embodiment of a FAST device 855. The FAST device comprises at least one illumination source 825, such as a laser source or the Ex illumination source that generates light that is transmitted through fibers. In alternate embodiments, the illumination source 825 is not limited and may comprise any of the alternative illumination sources described herein. The FAST device 855 comprises a two-dimensional end 856 and a one-dimensional end 857. In one embodiment, the two-dimensional end 856 has an ordering. The specific ordering of the two-dimensional end 856 is not limited. In some embodiments, the ordering is a serpentine ordering. The two-dimensional end 856 of the FAST device 855 comprises a two-dimensional array of optical fibers that are arranged into a one-dimensional fiber end. In one embodiment, the two-dimensional end 865 is non-linear. Such a non-linear configuration is not limited and can be one or more of circular, square, rectangular, and combinations thereof. Furthermore, in an embodiment, the one-dimensional end 857 is linear, forming a straight line.

At least a portion of the interacted photons from the sample tissue can be focused onto the input of the FAST device 855, which is the two-dimensional end 865. In some embodiments, the FAST device includes about 50, about 60, about 70, about 80, about 90, about 100, about 110, about 120, about 130, about 140, or about 150 FAST fibers. In one embodiment, there are less than about 100 FAST fibers. In another embodiment, there are about 96 FAST fibers. The number of FAST fibers can be in a range with any of the above numbers serving as an endpoint.

Referring again to FIG. 8, the one-dimensional fiber end 857 is oriented at the entrance slit of a spectrometer 860. The spectrometer 860 functions by separating the plurality of photons from the one-dimensional end 857 into a plurality of wavelengths and providing a separate dispersive spectrum from each fiber. Multiple Raman spectra and, therefore, multiple interrogations of the sample area can be obtained in a single measurement cycle. Inclusion of the FAST device permits the endoscope or endoscopic system to capture multiple Raman spectra in about the same amount of time that it takes for a conventional Raman sensor to collect one spectrum. Thus, the FAST device permits a considerable reduction in acquisition time. Photons may be detected at a detector 865 to generate to generate a Raman data set. In one embodiment, a processor (not shown) extracts spectral and/or spatial information that is embedded in a single frame that is generated by the detector 865. Although the detector 865 is depicted as a CCD, it is appreciated that any suitable detector can be selected, including the different kinds of camera chips and corresponding color filter arrays that are described above.

In FIG. 8, 861 is representative of detector 865 output, 862 is representative of an exemplary spectral reconstruction, and 863 is representative of an exemplary image reconstruction. In one embodiment, an area of interest can be optically matched by the FAST device to an area of a laser spot to maximize the collection Raman efficiency. In one embodiment, the present disclosure contemplates a configuration in which only the laser beam is moved for scanning within a field of view (FOV). The present disclosure also contemplates an embodiment in which the sample is moved and the laser beam is stationary.

It is possible to optically match the “scanning” FOV with the Raman collection FOV. The FOV is imaged onto a rectangular FAST device so that each FAST fiber is collecting light from one region of the FOV. The area per fiber which yields the maximum spatial resolution is easily calculated by dividing the area of the entire FOV by the number of fibers. Raman scattering is only generated when the laser excites a sample, so Raman spectra will only be obtained at those fibers whose collection area is being scanned by the laser beam. Scanning only the laser beam is a rapid process that may utilize off-the-shelf galvanometer-driven mirror systems.

The construction of the FAST device 855 requires knowledge of the position of each fiber at both the two-dimensional end 856 and the one-dimensional end 857 of the array. Each fiber collects light from a fixed position in the two-dimensional end 856 and transmits this light onto a fixed position on the detector 865 (through that fiber's one-dimensional end 857).

Each fiber may span more than one detector row, allowing higher resolution than one pixel per fiber in the reconstructed image. In fact, this super-resolution, combined with interpolation between fiber pixels (i.e., pixels in the detector associated with the respective fiber), achieves much higher spatial resolution than is otherwise possible. Thus, spatial calibration may involve not only the knowledge of fiber geometry (i.e., fiber correspondence) at the imaging end and the distal end, but also the knowledge of which detector rows are associated with a given fiber.

One of the fundamental advantages of using a FAST device over other spectroscopic methods is speed of analysis. A FAST device can acquire a few to thousands of full-spectral-range, spatially resolved spectra simultaneously. A complete spectroscopic imaging data set can be acquired in the amount of time it takes to generate a single spectrum from a given material using conventional means, especially for tissue samples that are susceptible to laser-induced photodamage. FAST devices can also be implemented with multiple detectors, and color-coded FAST spectroscopic images can be superimposed on other high-spatial resolution gray-scale images to provide significant insight into the condition and chemistry of the tissue sample.

Utilizing a FAST device is one way of configuring an endoscopic system for what may be referred to as “multipoint” analysis. To perform multipoint analysis, the tissue sample and field to be evaluated are illuminated in whole or in part, depending on the nature of the tissue sample and the type of multipoint sampling desired. A field of illumination can be divided into multiple adjacent, non-adjacent, or overlapping points, and spectra can be generated at each of the points. In one embodiment, these spectra may be averaged. In another embodiment, an illumination spot size can be increased sufficiently to spatially sample/average over a large area of the sample. This may also include transect sampling.

By way of example, the entire tissue sample can be illuminated and multipoint analysis can be performed by assessing interacted photons at selected points. Alternatively, multiple points of the tissue sample can be illuminated, and interacted photons emanating from those points can be assessed. The points can be assessed serially (i.e., sequentially). To implement this strategy, there is an inherent trade-off between acquisition time and the spatial resolution of the spectroscopic map. Each full spectrum takes a certain time to collect. As more spectra are collected per unit area of a sample, both the apparent resolution of the spectroscopic map and the data acquisition time increases. In another embodiment, interacted photons can be assessed in parallel (i.e., simultaneously) for all selected points in an image field. This parallel processing of all points is designated chemical imaging, and can require significant data acquisition time, computing time and capacity when very large numbers of spatial points and spectral channels are selected. However, chemical imaging may require less data acquisition time, computing time and capacity when a relatively small number of spectral channels are assessed.

In one embodiment, interacted photons may be assessed at multiple points in a FOV (e.g., the field of magnification for a microscope) that together represent only a portion of the area of the FOV (multipoint). It has been discovered that sampling the FOV at points representing a minority of the total area of the field (e.g., at two, three, four, six, ten, fifty, one hundred or more points and/or points representing, in sum, 25%, 5%, 1%, or less of the field) may provide a valuable representation of the FOV. The points can be single pixels of an image of the FOV or areas of the FOV represented in an image by multiple adjacent or grouped pixels. The shape of areas or pixels assessed as individual points is not critical. For example, circular, annular, square, or rectangular areas or pixels can be assessed as individual points. Lines of pixels may also be assessed in a line scanning configuration.

The area corresponding to each point of a multipoint analysis can be selected or generated in a variety of known ways. In one embodiment, structured illumination may be used. By way of example, a confocal mask or diffracting optical element placed in the illumination or collection optical path can limit illumination or collection to certain portions of the sample having a defined geometric relationship.

Spectroscopic analysis of multiple points in a FOV (multipoint analysis) allows high quality spectral sensing and analysis without the need to perform spectral imaging at every picture element (pixel) of an image. Optical imaging (e.g. RGB imaging) can be performed on the sample (e.g., simultaneously or separately), and the optical image can be combined with selected spectral information to define and locate regions of interest. Rapidly obtaining spectra from sufficiently different locations of this region of interest at one time allows highly efficient and accurate spectral analysis and the identification of components in samples. Furthermore, identification of a region of interest in a sample or in a FOV can be used as a signal that more detailed Raman scattering (or other) analysis of that portion of the sample or FOV should be performed.

The high number of optical fibers required for FAST spectroscopic and/or imaging applications places extraordinary demands on the imaging spectrograph, which the multipoint method addresses. Instead of having millions of pixels, multipoint analysis can utilize larger diameter fibers in bundles containing two to thousands of fibers. In the multipoint method of spectral sensing and analysis, complete spectral imaging (which would require at least thousands of adjacent pixels to create a physical image) is not required. Instead, spectral sensing performed at two to thousands of points simultaneously can rapidly (on the order of seconds) provide high quality spatially resolved spectra from a wide variety of points on the sample needed for analysis and identification. Thus, even if the precise geometric arrangement of the points analyzed in the FOV is not known, the points nonetheless have a defined geometric arrangement that can span a sample or a FOV. The analyzed points may be informative regarding the disease state of a tissue sample.

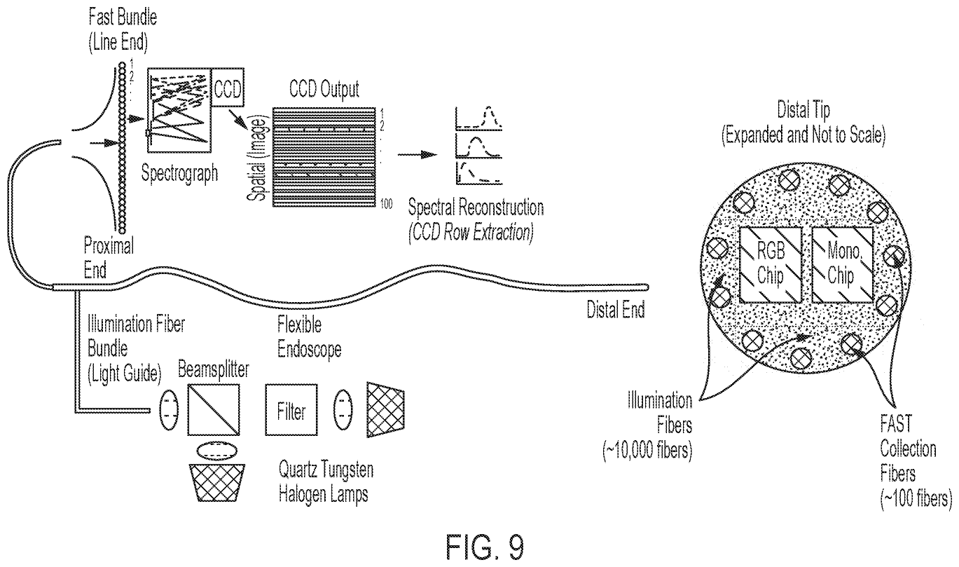

Referring now to FIG. 9, another embodiment is described that includes a flexible endoscope that combines illumination fibers, imaging fibers, and FAST fibers in a single endoscope. The endoscope that is depicted is a flexible endoscope that is suitable for insertion into body cavities and orifices; however, rigid endoscopes that are suitable for surgical manipulation through incisions are also contemplated. FIG. 9 depicts a flexible endoscope that includes a proximal end, which does not interact with the patient and which is connected to a spectrometer in the same manner described above with respect to FIG. 8. The exact parts and components will not be repeated here. The flexible endoscope also include a distal end that is intended to be inserted within one or more of a body orifice, a body cavity, an incision, and the like and combinations thereof. On the proximal end, in addition to the FAST device, a filtered illumination source is provided in the form of quartz tungsten halogen lamps, lasers, or both. As will be appreciated by those of skill from reading the above disclosure, the illumination source is not particularly limited and the selection of the illumination source is described above.

Referring again to FIG. 9, the endoscope also includes a distal end that is configured to be inserted into a patient for a surgical procedure, to assist with diagnostics, and combinations thereof. Also, as described above, the distal end in some embodiments includes one or more of illumination fibers, FAST fibers, and camera chips. It is appreciated that the endoscope described in FIG. 9 and which forms an endoscopic system of the disclosure is not limited in configuration and can include any of the combinations of illumination fibers, FAST fibers, and camera chips that are described throughout the application.

In some embodiments, the endoscopic system has a refresh rate of at least about 1 frame per second (fps), at least about 2 fps, at least about 3 fps, at least about 4 fps, at least about 5 fps, at least about 6 fps, at least about 7 fps, at least about 8 fps, at least about 9 fps, at least about 10 fps, at least about 11 fps, at least about 12 fps, at least about 13 fps, at least about 14 fps, or at least about 15 fps.

The variations as described in reference to FIGS. 1-3 and 5-9 above can be implemented using one of the two optical configuration options shown in FIG. 4. A first optical configuration, labeled Option 1 in FIG. 4, includes an illumination source 401 directed at a beamsplitter 402. The beamsplitter 402 can be configured to split the beam into two beams such that each of the split beams includes about 50% of the original light emitted by the illumination source 401. A first split light beam is directed through the Ex source illumination path, and a second split light beam is directed through the T1 and T2 source illumination path. Following the Ex source illumination path, the first split light beam can be directed through a modulator 403, and reflected by a mirror 404 to an excitation filter 405. The output of the excitation filter 405 can pass through a fiber coupling lens 406 and be output through the Ex source illumination optical fiber bundle 407. Following the T1 and T2 path, the second split light beam can pass through a second beamsplitter 408. The output of the second beamsplitter 408 can be two equal beams, now each approximately 25% of the total light emitted by the illumination source 401. The first beam can pass through a modulator 409, be reflected by a mirror 410, and filtered by a filter 411. The filtered beam can pass through a fiber coupling lens 412 and be output through the T1 source illumination optical fiber bundle 413. The second beam (from beamsplitter 408) can pass through a modulator 414 and be filtered by a filter 415. The filtered beam can pass through a fiber coupling lens 416 and be output through the T2 source illumination optical fiber bundle 417.

By actively controlling the operation of one or more of the modulators 403, 409, and 414, the output of the configuration as shown in Option 1 can be accurately controlled. For example, by activating modulators 409 and 414, and deactivating modulator 403, both T1 and T2 can actively output source illumination. Similarly, by activating modulator 403 and deactivating modulators 409 and 414, Ex can actively output source illumination.

A second optical configuration, labeled Option 2 in FIG. 4, includes an illumination source 421 directed at a movable mirror 422. Depending upon the position of the movable mirror 422, the light emitted from the illumination source 421 can travel either the Ex source illumination path (represented by the solid line in Option 2) or the T1 and T2 source illumination path (represented by the dashed line in Option 2).

Following the Ex source illumination path, the light reflected by the movable mirror 422 can be further reflected by a mirror 423 to an excitation filter 424. The output of the excitation filter 424 can pass through a fiber coupling lens 425 and be output through the Ex source illumination optical fiber bundle 426.

If the movable mirror 422 is positioned such that the light emitted by the illumination source 421 is not reflected, the light can follow the T1 and T2 path. The light beam can pass through a beamsplitter 427. The output of the beamsplitter 427 can be two equal beams, now each approximately 50% of the total light emitted by the illumination source 421. The first beam (from beamsplitter 427) can pass through a modulator 428, be reflected by a mirror 429, and be filtered by a filter 430. The filtered beam can pass through a fiber coupling lens 431 and be output through the T1 source illumination optical fiber bundle 432. The second beam (from beamsplitter 427) can pass through a modulator 433 and be filtered by a filter 434. The filtered beam can pass through a fiber coupling lens 435 and be output through the T2 source illumination optical fiber bundle 436.

By actively controlling the position of the movable mirror 422, as well as the operation of one or more of the modulators 428 and 433, the output of the configuration as shown in Option 2 can be accurately controlled. For example, by moving movable mirror 422 into position to reflect the light emitted by illumination source 421, all emitted light can be directed to the Ex optical fiber bundle 426. Similarly, by positioning the movable mirror 422 into a position where no light emitted by the illumination source 421 is reflected, and by actively controlling modulators 428 and 433, light can be output to one or more of the T1 source illumination optical fiber bundle 432 and the T2 source illumination optical fiber bundle 436.

The table in FIG. 4 compares the relative illumination intensity throughput and other performance metrics of configuration Options 1 and 2. However, it should be noted that the specific values contained in the table are relevant to the particular designs included in Options 1 and 2, which are provided by way of example only. Depending upon the design of and implementation of an endoscopy system, the designs shown in Options 1 and 2 can be modified accordingly. For example, depending upon the available space for circuit implementation, the number of and/or position of the mirrors in the configurations may be altered. Additionally, in certain implementations, the function of the movable mirror 422 of Option 2 may be altered. For example, when positioned to reflect light, the movable mirror 422 may be configured to direct the reflected light to the T1 and T2 pathway while non-reflected light (i.e., the movable mirror 422 is positioned where light emitted from the illumination source 421 is not reflected) travels down the Ex pathway.

In some embodiments, the chip-on-tip product or the endoscopic system of is used as part of a method of generating a fused image. In such embodiments, a first plurality of modulated photons are used to generate a first image, and a second plurality of modulated photons are used to generate a second image. The first image and the second image are used to generate a fused image. In some embodiments, additional images beyond the first image and the second image are generated, and the additional images may be generated from modulated and/or unmodulated photons. Each of the first, second and additional images may be generated from photons in the ranges of ultraviolet (UV), visible (VIS), near infrared (NIR), visible-near infrared (VIS-NIR), shortwave infrared (SWIR), extended shortwave infrared (eSWIR), near infrared-extended shortwave infrared (NIR-eSWIR). In some embodiments, the plurality of unmodulated photons are NIR, SWIR, or eSWIR photons. In other embodiments, a first plurality of modulated photons are VIS photons, and a second plurality of modulated photons are VIS-NIR photons.

In the above detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be used, and other changes may be made, without departing from the spirit or scope of the subject matter presented herein. It will be readily understood that various features of the present disclosure, as generally described herein, and illustrated in the Figures, can be arranged, substituted, combined, separated, and designed in a wide variety of different configurations, all of which are explicitly contemplated herein.

The present disclosure is not to be limited in terms of the particular embodiments described in this application, which are intended as illustrations of various features. Many modifications and variations can be made without departing from its spirit and scope, as will be apparent to those skilled in the art. Functionally equivalent methods and apparatuses within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is to be understood that this disclosure is not limited to particular methods, reagents, compounds, compositions or biological systems, which can, of course, vary. It is also to be understood that the terminology used herein is for the purpose of describing particular embodiments only, and is not intended to be limiting.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (for example, bodies of the appended claims) are generally intended as “open” terms (for example, the term “including” should be interpreted as “including but not limited to,” the term “having” should be interpreted as “having at least,” the term “includes” should be interpreted as “includes but is not limited to,” et cetera). While various compositions, methods, and devices are described in terms of “comprising” various components or steps (interpreted as meaning “including, but not limited to”), the compositions, methods, and devices can also “consist essentially of” or “consist of” the various components and steps, and such terminology should be interpreted as defining essentially closed-member groups. It will be further understood by those within the art that if a specific number of an introduced claim recitation is intended, such an intent will be explicitly recited in the claim, and in the absence of such recitation no such intent is present.

For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (for example, “a” and/or “an” should be interpreted to mean “at least one” or “one or more”); the same holds true for the use of definite articles used to introduce claim recitations.

In addition, even if a specific number of an introduced claim recitation is explicitly recited, those skilled in the art will recognize that such recitation should be interpreted to mean at least the recited number (for example, the bare recitation of “two recitations,” without other modifiers, means at least two recitations, or two or more recitations). Furthermore, in those instances where a convention analogous to “at least one of A, B, and C, et cetera” is used, in general such a construction is intended in the sense one having skill in the art would understand the convention (for example, “a system having at least one of A, B, and C” would include but not be limited to systems that have A alone, B alone, C alone, A and B together, A and C together, B and C together, and/or A, B, and C together, et cetera). In those instances where a convention analogous to “at least one of A, B, or C, et cetera” is used, in general such a construction is intended in the sense one having skill in the art would understand the convention (for example, “a system having at least one of A, B, or C” would include but not be limited to systems that have A alone, B alone, C alone, A and B together, A and C together, B and C together, and/or A, B, and C together, et cetera). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, et cetera. As a non-limiting example, each range discussed herein can be readily broken down into a lower third, middle third and upper third, et cetera. As will also be understood by one skilled in the art all language such as “up to,” “at least,” and the like include the number recited and refer to ranges that can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

Various of the above-disclosed and other features and functions, or alternatives thereof, may be combined into many other different systems or applications. Various presently unforeseen or unanticipated alternatives, modifications, variations or improvements therein may be subsequently made by those skilled in the art, each of which is also intended to be encompassed by the disclosed embodiments.

What is claimed is:

1. A hybrid imaging product for use in an endoscope, the hybrid imaging product comprising:
a first plurality of source illumination fibers configured to transmit a first plurality of modulated photons;
a second plurality of source illumination fibers configured to transmit a second plurality of modulated photons;
a plurality of fiber array spectral translator (FAST) fibers; and
a first image collector that includes one or more of imaging fibers or a camera sensor.

2. The hybrid imaging product of claim 1, further comprising a third plurality of source illumination fibers configured to transmit a third plurality of unmodulated photons.

3. The hybrid imaging product of claim 1, further comprising a second image collector, wherein the second image collector includes one or more of imaging fibers or a camera sensor.

4. An endoscopic system, the endoscopic system comprising:
a hybrid imaging product that includes:
a first plurality of source illumination fibers configured to transmit a first plurality of modulated photons,
a second plurality of source illumination fibers configured to transmit a second plurality of modulated photons,
a plurality of fiber array spectral translator (FAST) fibers, and
a first image collector that includes one or more of imaging fibers or a camera sensor;
an illumination source;
a first modulator that modulates at least the first plurality of modulated photons for the first plurality of source illumination fibers; and
a second modulator that modulates at least the second plurality of modulated photons for the second plurality of source illumination fibers.

5. The endoscopic system of claim 4, wherein the hybrid imaging product further comprises a third plurality of source illumination fibers configured to transmit a third plurality of unmodulated photons.

6. The endoscopic system of claim 4, wherein the hybrid imaging product further includes a second image collector, and the second image collector includes one or more of imaging fibers or a camera sensor.

7. The endoscopic system of claim 4, wherein the illumination source includes an incandescent lamp, halogen lamp, light emitting diode (LED), quantum cascade laser, quantum dot laser, external cavity laser, chemical laser, solid state laser, organic light emitting diode (OLED), electroluminescent device, fluorescent light, gas discharge lamp, metal halide lamp, xenon arc lamp, induction lamp, or combinations thereof.

8. The endoscopic system of claim 4, wherein the first modulator or the second modulator is each independently one or more of an acousto-optic tunable filter (AOTF), a liquid crystal tunable filter (LCTF), a multivariate optical element (MOE), a filter wheel, a patterned etalon filter, a multi-conjugate filter (MCF), or a conformal filter (CF).

9. A method of generating a fused image using an endoscopic system that includes a hybrid imaging product that includes a first plurality of source illumination fibers configured to transmit a first plurality of modulated photons, a second plurality of source illumination fibers configured to transmit a second plurality of modulated photons, a plurality of fiber array spectral translator (FAST) fibers; and a first image collector that includes one or more of imaging fibers or a camera sensor; an illumination source; a first modulator that modulates at least the first plurality of modulated photons for the first plurality of source illumination fibers; and a second modulator that modulates at least the second plurality of modulated photons for the second plurality of source illumination fibers, the method comprising:
generating a first image from a first plurality of modulated photons;
generating a second image from a second plurality of modulated photons; and
overlaying the first image and the second image to thereby generate a fused image.

10. The method of claim 9, further comprising:
generating a third image from a third plurality of unmodulated photons.

11. The method of claim 9, wherein the third plurality of unmodulated photons are NIR photons, SWIR photons, eSWIR photons, or combinations thereof.

12. The method of claim 9, wherein each of the first plurality of modulated photons and the second plurality of modulated photons are independently VIS or VIS-NIR.
